(19) United States
(12) Patent Application Publication
(10) Pub. No.: US 2025/0352495 A1
(43) Pub. Date: Nov. 20, 2025

(54) HYDROGEL-COATED PENIS PATCH FOR SEX PROLONGATION AND PREMATURE EJACULATION RELIEF

(71) Applicants: XUANSHUO ZHANG, PLANO, TX (US); YIHAN ZHANG, PLANO, TX (US)

(72) Inventors: XUANSHUO ZHANG, ZHOUKOU (CN); YIHAN ZHANG, CHANGZHOU (CN)

(21) Appl. No.: 18/664,235

(22) Filed: May 14, 2024

(51) Int. Cl. A61K 9/70; A61F 5/41; A61K 9/00; A61K 9/06; A61K 31/167; A61K 31/245; A61K 31/522; A61K 36/185; A61K 36/234; A61K 36/41; A61K 36/61

(52) U.S. Cl. CPC A61K 9/7092; A61F 5/41; A61K 9/0034; A61K 9/06; A61K 31/167; A61K 31/245; A61K 31/522; A61K 36/185; A61K 36/234; A61K 36/41; A61K 36/61

(57) ABSTRACT

This invention presents a disposable male hydrogel patch designed to delay ejaculation and prolong erection time while minimizing the impact on sexual pleasure. The patch features an ergonomically designed backing with specific curvatures, a drug-loaded strip divided into central and side zones, and an optional hydrogel coating for sustained drug release. The central zone contains a drug mixture that inhibits the dorsal nerve of the penis, while the side zones promote vasodilation related to erection. The backing includes the adjustment hole for adaptive fitting and is made of a multi-layer composite that integrates breathability, waterproofing, moisture absorption, and fixation. The drug-loaded strip incorporates herbal extracts and synthetic compounds to target the nervous system, vascular system, and endocrine system. Discrete protruding arrays at the coronal sulcus recess enhance drug delivery. This novel, multi-angle approach comprehensively improves sexual function and quality of life, making it a significant innovation in male sexual health.

### FIELD OF THE INVENTION

The present invention relates to a disposable male hydrogel patch for delaying ejaculation and improving erectile function. The patch employs ergonomic design and a unique drug formulation, integrating a backing, a drug-loaded strip, and a hydrogel coating to achieve differentiated drug delivery to different regions of the penile coronal sulcus, thereby delaying ejaculation, prolonging erection time, and minimizing the impact on sexual pleasure.

### BACKGROUND OF THE INVENTION

The desire to prolong sexual intercourse has been a concern for men since ancient times. For example, the ancient Indian text “Kama Sutra” recorded techniques for enhancing sexual endurance and delaying ejaculation. In modern times, premature ejaculation (PE) is a common problem, affecting approximately 30% of males worldwide. In addition to physical effects, PE can lead to psychological and emotional issues, such as low satisfaction, decreased self-esteem, and anxiety. It can also strain intimate relationships, leading to inter-personal problems and reduced intimacy, forming a vicious cycle.

Various treatment methods have been employed to increase the duration of sexual intercourse for men, including oral medications, topical treatments, behavioral techniques, and surgical interventions.

Oral medications are widely used for PE treatment, such as selective serotonin reuptake inhibitors (SSRIs). For example, dapoxetine has been approved in many countries for the treatment of PE. Other SSRIs, such as fluoxetine (U.S. Pat. No. 4,536,518) and paroxetine (U.S. Pat. No. 4,940,731), have also been found to effectively delay ejaculation by regulating serotonin levels in the brain, although these patents primarily focus on their applications in treating depression and other conditions. In addition to SSRIs, phosphodiesterase type 5 inhibitors (PDE-5Is) such as sildenafil (Viagra, U.S. Pat. No. 5,250,534), tadalafil (Cialis, U.S. Pat. No. 6,140,329), and vardenafil (Levitra, U.S. Pat. No. 6,469,012 B1) have also been used to treat PE, although researchers primarily focus on their applications in treating erectile dysfunction. Tramadol, a weak opioid analgesic, has also been used to delay ejaculation. However, these medications may be associated with various side effects, such as nausea, dizziness, xerostomia, and decreased libido. Moreover, not all patients are suitable for using these medications, especially those with cardiovascular, liver, or kidney complications. This highlights the importance and necessity of developing novel, safe, and effective treatments for PE.

Topical treatments in the form of anesthetic sprays or creams are increasingly popular, often containing anesthetics such as lidocaine or benzocaine to prolong ejaculation time by reducing the sensitivity of the glans penis. However, the timing and application technique of the medication need to be correctly grasped, meaning that it may be difficult to achieve optimal anesthetic distribution on the penis. Secondly, excessive use of anesthetics may lead to erectile dysfunction or other side effects. Additionally, the anesthetic effect may extend to the sexual partner, resulting in reduced sexual sensitivity for both parties. There is also a risk of allergic reactions or local skin irritation when using topical anesthetics.

Behavioral techniques, such as the “stop-start” method and the “squeeze” technique, can effectively delay ejaculation but require practice, communication, and cooperation between partners. However, long-term efficacy may be limited, and these techniques may not address underlying psychological or biological factors.

Although surgical interventions are uncommon, selective dorsal nerve neurectomy has been explored as a method to reduce penile sensitivity and delay ejaculation. However, surgery is invasive and irreversible, and it may carry risks such as paralysis, pain, and erectile dysfunction. The long-term efficacy and safety of surgical interventions remain controversial.

In recent years, there has been growing interest in exploring natural and herbal therapies for the treatment of PE. Traditional herbal extracts, such as Tribulus terrestris, Withania somnifera, and Asparagus racemosus, are widely believed to act through multiple mechanisms, such as increasing testosterone levels, improving erectile function, and regulating ejaculatory neurotransmitters. However, evidence for herbal therapies is limited, and further research is needed to determine their therapeutic potential and optimal formulations. At the same time, many individuals struggling with PE seek solutions through the internet, but the quality and reliability of online information and products are questionable. Many unverified advertisements claim to contain natural ingredients that can enhance sexual performance and delay ejaculation, but these products may contain undisclosed ingredients and lack proper safety testing.

Hydrogels are three-dimensional networks of hydrophilic polymers that can absorb and retain large amounts of water and release drugs in an orderly manner, making them potentially excellent carriers for drug delivery and tissue engineering applications. They have been widely used in areas such as face masks and drug-loaded scaffolds. The unique properties of hydrogels, such as biocompatibility, flexibility, and the ability to incorporate various drugs or bioactive molecules, make them ideal promising materials for developing novel therapeutic systems. In the context of PE, hydrogel-based formulations may provide local, controlled release of active ingredients to the penis while minimizing systemic absorption and reducing the risk of adverse reactions. Furthermore, the moisturizing properties of hydrogels can help maintain natural lubrication and reduce friction during sexual intercourse, further enhancing the user experience.

The human penis has a relatively complex topological surface. Simply using a rectangular patch cannot conform to the non-uniformly changing curvature of the coronal sulcus surface in space. Ergonomics and computer-simulated discrete differential geometry provide a framework for analyzing and describing complex surfaces and shapes, considering their curvature, thickness, and other geometric properties. These principles have been applied in various fields, including product design, architecture, and biomedical engineering, to create more comfortable, functional, and aesthetically pleasing objects and structures. By considering the unique anatomical contours and surface characteristics of the penis, the present invention utilizes computer simulation to design a patch that closely conforms to the coronal sulcus surface, maximizing contact with the penile skin while minimizing discomfort or interference with sexual activity.

### SUMMARY OF THE INVENTION

The present invention relates to a disposable male hydrogel patch for delaying ejaculation and prolonging erection time while minimizing the impact on sexual pleasure. The patch consists of a backing, a drug-loaded strip, and a hydrogel coating.

The patch adopts a 3D ergonomic design with specific curvatures that can perfectly conform to the penile coronal sulcus, allowing the active ingredients to precisely target the dorsal nerve of the penis (DNP). The drug-loaded strip is divided into a central zone and side zones. The central zone is loaded with a first drug mixture that inhibits the dorsomedial branches of the DNP, which are closely associated with ejaculation and are predominantly located in this area. The side zones are loaded with a second drug mixture that promotes vasodilation related to erection and improves blood circulation. The hydrogel coating covers the drug-loaded strip, controlling the sustained release of drug components, achieving stable and smooth release, and avoiding burning or freezing sensations that may occur when directly applied to the penile surface.

The right-side tail end of the backing has an adjustment hole for adaptive connection to the left-side tail end, allowing for free sliding to accommodate different states of erection and flaccidity. The backing adopts a multi-layer composite structure that integrates breathability, waterproofing, moisture absorption, and fixation functions. The drug-loaded strip is made of materials with good elasticity, flexibility, hydrophilicity, adsorption capacity, and biocompatibility.

The hydrogel encapsulation of the drug-loaded strip ensures comfort even during long-term wear. The incorporated natural herbal formula is gentle and has no side effects. Through herbal network pharmacology, the use of single ingredients can be reduced, achieving a compound additive effect and reducing side effects caused by high concentrations of single ingredients. The components can penetrate the skin and be absorbed to exert their effects.

The first drug mixture in the central zone contains natural ingredients like Cnidium monnieri extract and clove extract, or synthetic anesthetics like benzocaine and lidocaine. The second drug mixture in the side zones contains damiana extract, Rhodiola rosea extract, Erythroxylum extract, caffeine, and theophylline.

Discrete hemispherical, semi-cylindrical, or semi-vertebral protruding arrays are set at the coronal sulcus recess of the drug-loaded strip. The hydrogel coating is prepared using natural polysaccharides, synthetic polymers, or amphoteric ion polymers.

According to the preferred embodiment of the invention, the user takes out the disposable male hydrogel patch 30 minutes to 12 hours before sexual intercourse. Align the inner side of the central zone with the dorsal part of the coronal sulcus and wrap the hemispherical protruding array around the penis in a flaccid state along the coronal sulcus for one circle. Leave the patch in place for at least 30 minutes to allow the natural herbal ingredients to fully diffuse into the penile epidermis, then remove it.

The disposable male hydrogel patch of the present invention utilizes a multi-angle and multi-level strategy to improve the user's sexual function and quality of life by regulating the nervous system, vascular system, and endocrine system, which is a major innovation and advantage of this invention.

### BRIEF DESCRIPTION OF THE DRAWINGS

For a more complete understanding of the invention, reference is made to the following description and accompanying drawings, in which:

FIG. 1 illustrates the top view of the hydrogel penis patch laid flat on a horizontal surface;

FIG. 2 illustrates the bottom view of the hydrogel penis patch laid flat on a horizontal surface;

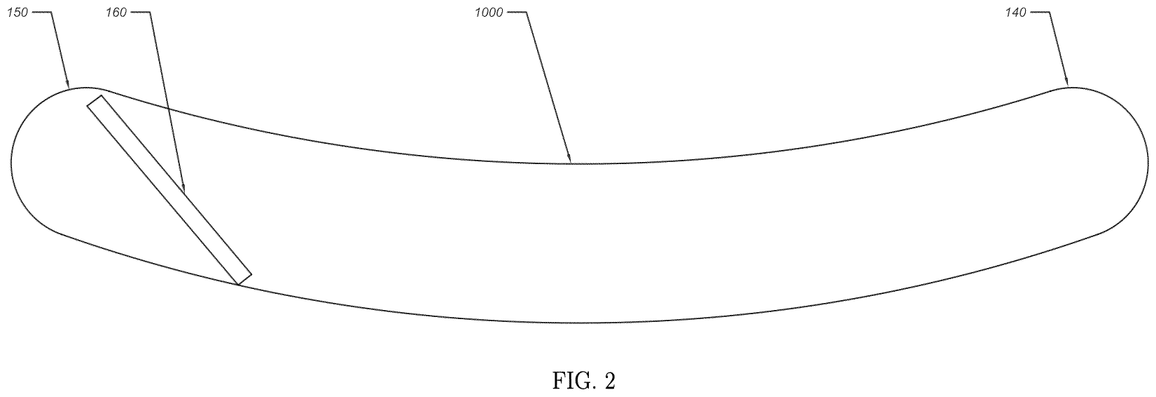

FIG. 3 illustrates the side view of a penis with a hydrogel patch attached;

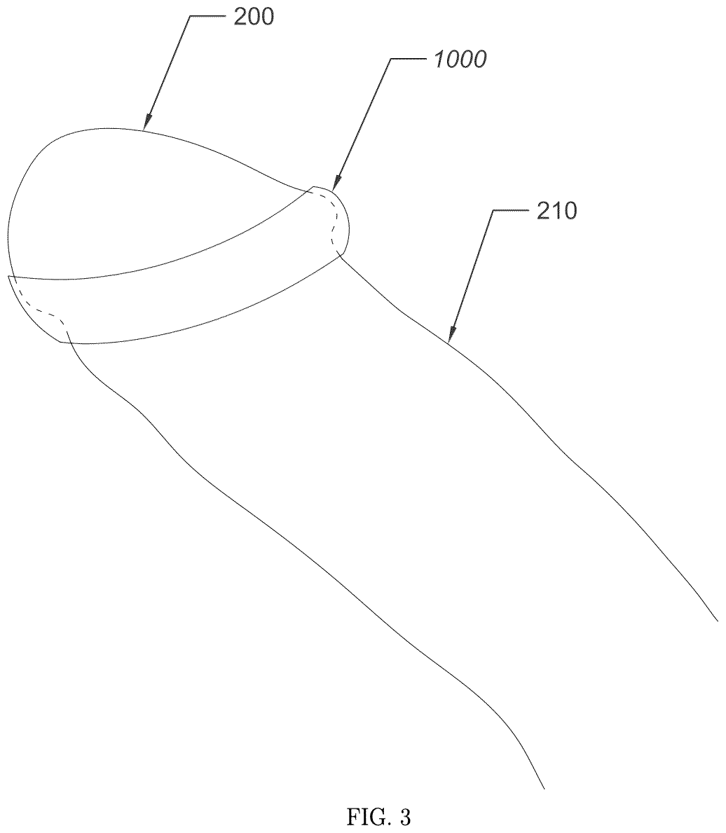

FIG. 4 illustrates the bottom view of the penis with a hydrogel patch attached, with two types of adjustment holes, the internal type and the external type;

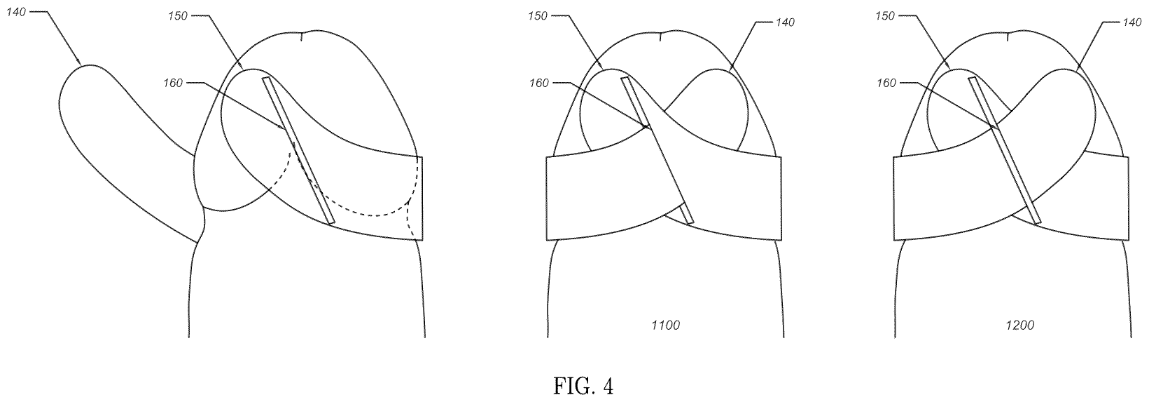

FIG. 5 illustrates the bottom view of the penis with a hydrogel patch attached. The Magnified view illustrates the fit of the hemispherical protrusion into the groove of the coronal sulcus. The Magnified illustrates the structure of the patch package.

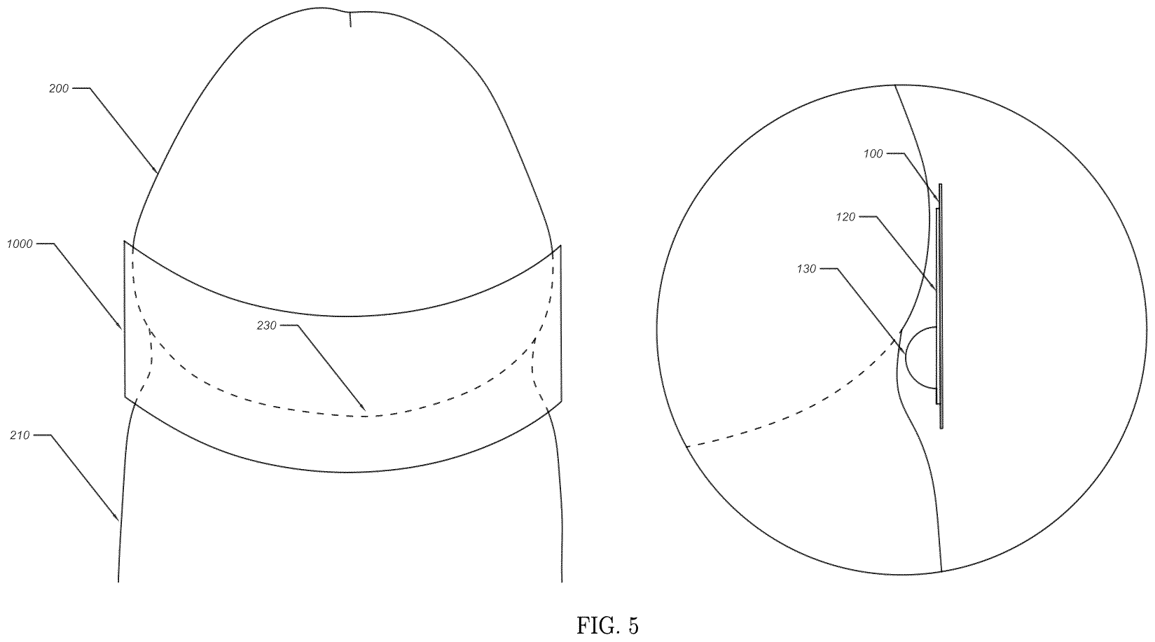

### DETAILED DESCRIPTION OF THE PREFERRED EMBODIMENTS

According to the present invention, the disposable male hydrogel patch 1000 is equipped with two zones: a central zone 110 and side zones 120, which can delay male ejaculation, help prolong penile erection time, and simultaneously affect sexual pleasure to a lesser extent.

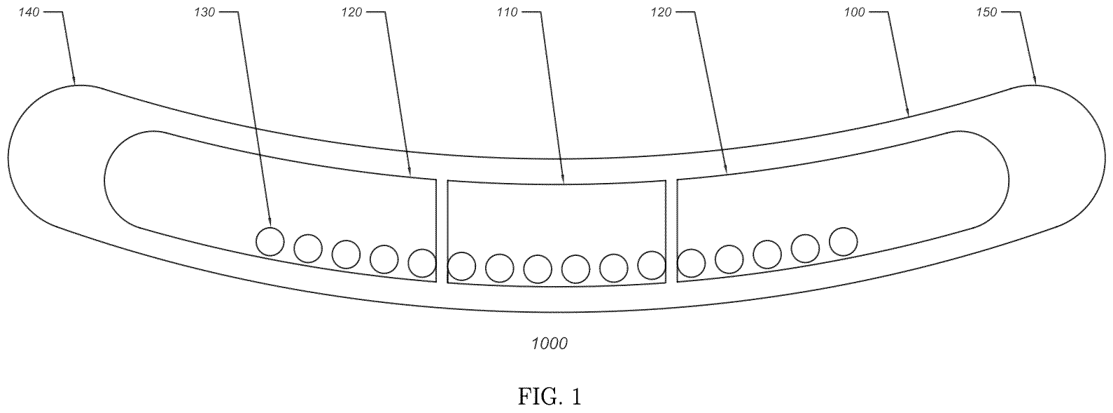

FIG. 1 shows a top view of the hydrogel patch 1000. The patch 1000 contains: a backing 100 with specific curvatures based on ergonomic design, drug-loaded strips 105 in different zones including a central zone 110 and side zones 120, a protruding array 130 that is easy to fold in space, a hydrogel coating 400 covering the drug-loaded strips 105, and an adjustment hole 160 on the right tail end for connection.

30 minutes to 12 hours before sexual activity, the user takes the disposable hydrogel patch 1000 out of the package, aligns the inner side of the central zone 110 of the patch 1000 with the dorsal side of the middle of the coronal sulcus 230, and wraps the hemispherical protruding array 130 around the penis in a flaccid state along the coronal sulcus 230. When the patch 1000 fits tightly with the coronal sulcus 230, the left tail end 140 of the patch is inserted into the adjustment hole 160 of the right tail end 150 and adjusted to a position suitable for the user's penis size. It is left in place for at least 30 minutes, allowing the natural herbal ingredients 300 to fully diffuse to the penile epidermis, and then the patch 1000 is removed.

In the present invention, the adjustment hole 160 on the right tail end 150 of the patch allow the left tail end 140 to actively slide and adapt to the current size, thus accommodating the change in the circumference of the coronal sulcus brought about by the transition of the penis from flaccid to erect state. Due to the certain adhesiveness of the hydrogel itself and the ability of the internal protruding structure to embed into the curved groove of the coronal sulcus, the risk of patch displacement is reduced. This sliding structure can be represented as an embedded type 1100 and an externally embedded type 1200. The embedded type 1100 is directly cut into the right tail end 150, while the externally embedded type 1200 is formed by stitching on the right tail end 150.

The patch 1000 of the present invention can also include the following tail end connection designs: double-sided adhesive bonding, memory metal, magnetic attraction, Velcro connection, buttons or buckles, zippers, knots, leather straps, etc. Compared to adjustment holes 160, the disadvantages of these designs are higher cost or difficulty in automatically adapting to changes in the circumference of the coronal sulcus brought about by erection. It should be emphasized that even without a tail end connection structure, the coronal sulcus patch of the present invention can still be attached to the surface of the penis. Therefore, the scope of protection of the present invention covers all the above-mentioned tail end connection designs and designs without connection. Any coronal sulcus patch that adopts one or more of these designs, or variations or combinations based on these designs, falls within the scope of protection of this patent.

According to the preferred embodiment of the present invention, the backing 100 can use a multi-layer composite material structure that integrates the functions of breathability, waterproofing, moisture absorption, and fixation. In some embodiments, the backing 100 can be made of materials such as polyurethane film, non-woven fabric, pressure-sensitive adhesive, release paper, mesh polyethylene film, etc. As shown in FIG. 1, the right tail end 150 of the backing 100 contains an adjustment hole 160. In order to better fit the coronal sulcus, the upper and lower edge curves of the backing 100 adopt an ergonomic design. Specifically, the present invention adopts a computer-simulated discrete differential geometry method to flatten the 3D modeled coronal sulcus surface to a 2D plane to obtain a base shape that better fits the coronal sulcus 230. In some embodiments, the total length of the backing 100 is approximately 6-9 cm. In some embodiments, the total length of the backing 100 is approximately 9-12 cm. In some embodiments, the total length of the backing 100 is approximately 12-15 cm.

The drug-loaded strip 105 is made of materials with proper elasticity, flexibility, hydrophilicity, adsorption capacity, and biocompatibility, such as special textile cotton, medical degreased cotton, and sticky fibers. As shown in FIG. 1, the drug-loaded strip 105 is divided into a central zone 110 and side zones 120. The covered area of the central zone 110 contains a large number of dorsomedial branches of the dorsal nerve of the penis (DNP), which have been proven by research to be closely related to ejaculation and ejaculation desire. By encapsulating the drug mixture 310 in the central zone 110, the dorsal nerve is inhibited, thereby delaying the generation of ejaculation desire. The diffusion range of the side zones 120 covers a small number of dorsomedial branches of DNP and a large number of lateral branches of DNP related to erection, and there are also more blood vessels. By loading the drug mixture 320 in the side zones 120, the effect of activating erection-related vascular receptors and dilating blood vessels is achieved, thereby helping users obtain a better erection experience.

In some embodiments, the drug mixture 310 in the central zone contains natural herbal ingredients that inhibit the dorsal nerve of the penis, such as osthole and clove extract. The concentration of natural herbal ingredients is generally selected to be 50 mg/mL to 300 mg/mL. In addition to the osthole extract and clove extract listed above, the present invention also covers other natural or synthetic nerve inhibitors with similar pharmacological effects and mild side effects as alternative components. These alternative components can exert the effects of delaying ejaculation, relieving premature ejaculation, and promoting testosterone secretion through mechanisms similar to the main components, such as antibacterial and anti-inflammatory effects, anesthesia and sedation, improving local blood circulation, and regulating neurotransmitters.

In some embodiments, the drug mixture 310 in the central zone may also contain a certain concentration of synthetic local anesthetics, such as benzocaine, lidocaine, etc. Among them, benzocaine is a commonly used local anesthetic drug that blocks sodium channels at nerve endings, inhibits the transmission of nerve impulses, and thereby reduces the sensory sensitivity of the penis. Clinical studies have shown that topical benzocaine can effectively prolong the ejaculation latency of patients with premature ejaculation and improve the quality of sexual life. The concentration of synthetic local anesthetic drugs is generally selected to be 10 mg/mL to 50 mg/mL. Combined with natural nerve inhibitors, it can further enhance the sedative and anesthetic effects, improve the ability to delay ejaculation, and have good tolerability. In the future, with the deepening of understanding of the pathogenesis of premature ejaculation and the progress of new drug research and development technology, the alternative components of the present invention may be further expanded and optimized.

The selection of components for the drug mixture 320 in the side zones is based on the following known pharmacological conclusions. First, flavonoids and terpenoids have sedative and anti-anxiety effects, which can help alleviate anxiety and nerve hypersensitivity, thereby prolonging ejaculation time. Therefore, in some embodiments, the drug mixture 320 in the side zones may contain ingredients with flavonoids and terpenoids, such as damiana extract and Rhodiola rosea extract. Second, elevated levels of the male hormone testosterone can promote vasodilation, enhance blood flow in the penis, and improve erection quality. Therefore, in some embodiments, the drug mixture 320 in the side zones may contain ingredients that promote elevated testosterone levels, such as Rhodiola rosea extract and Erythroxylum extract. Third, components such as serotonin and dopamine can affect the balance of neurotransmitters, stimulate and enhance the brain's response to sexual desire, thereby achieving emotional regulation and improving sexual function. Therefore, in some embodiments, the drug mixture 320 in the side zones may contain ingredients that promote elevated levels of components such as serotonin and dopamine, such as Erythroxylum extract. Fourth, methylxanthine alkaloids have the effect of exciting the nervous system, enhancing the perception and conduction of sexual stimulation, and thus delaying ejaculation time. At the same time, they can inhibit PDE5 and promote the relaxation of smooth muscles in the penile cavernous body, thereby improving erection quality. Therefore, in some embodiments, the drug mixture 320 in the side zones may contain ingredients with methylxanthine alkaloids, such as caffeine and theophylline. The concentration of natural herbal ingredients in the drug mixture 320 in the side zones is generally selected to be 20 mg/mL to 200 mg/mL. These components can be used alone or in combination at different ratios to obtain the best clinical effects.

In some embodiments, the drug mixture 320 in the side zones contains natural herbal ingredients with the above-mentioned pharmacological functions, such as damiana extract, Rhodiola rosea extract, Erythroxylum extract, caffeine, and theophylline. In some embodiments, the drug mixture 320 in the side zones may also include other natural plant extracts with similar pharmacological effects, such as ginseng, epimedium, and Cistanche deserticola.

The drug mixture 320 in the side zones of the present invention works in coordination with the drug mixture 310 in the central zone, starting from multiple aspects such as nerves, blood vessels, and endocrine, to comprehensively improve the user's sexual function and enhance their quality of life. This multi-angle and multi-level strategy is a major innovation and advantage of the present invention.

In some embodiments, the gap between the central zone drug-loaded strip 110 and the side zone drug-loaded strip 120 is within 6 mm. In some embodiments, there is no separation design between the central zone drug-loaded strip 110 and the side zone drug-loaded strip 120.

In some embodiments, the drug-loaded strip 100 includes a protruding array 160 at the coronal sulcus recess. This protruding array is generally discrete rather than continuous, and common types include semi-cylindrical arrays, hemispherical arrays, smooth semi-vertebral arrays, etc. This array serves three purposes. First, the protruding structure can better adapt to the recess of the coronal sulcus, thus making drug diffusion more uniform and controllable. Second, the non-continuous discrete design eliminates potential wrinkles brought by the continuous protruding structure and prevents concentration gradients caused by wrinkles during the bending process of the patch 1000. Finally, this 3D embedded structure can help reduce the risk of displacement of the patch 1000, thus ensuring that the central zone 110 can continuously optimize the effect on the dorsal nerve of the penis. In some embodiments, the protruding array can adopt a hemispherical protruding array 160, as shown in FIG. 1. Due to the isotropic geometric properties of the sphere, the hemispherical array 160 has the optimal spatial extensibility. In some embodiments, the drug-loaded strip 100 does not include a protruding array 160.

In some embodiments, the drug-loaded strip 100 is loaded with a hydrogel 400 that has excellent extensibility, high hydrophilicity, temperature sensitivity, and can achieve controlled and uniform release through a reasonable affinity with drug components. This hydrogel coating can achieve uniform or slow release of the drug mixture 300, thereby reducing skin irritation. Hydrogel materials can include natural polysaccharides, synthetic polymers, and amphoteric ion polymers.

In some embodiments, the hydrogel 400 can be selected from natural polysaccharides, including chitin and its derivatives, such as chitosan, carboxymethyl chitosan, etc. In some embodiments, the hydrogel 400 can be selected from alginates, such as sodium alginate, calcium alginate, etc. In some embodiments, the hydrogel 400 can be selected from natural macromolecular proteins, such as silk fibroin. In some embodiments, the hydrogel 400 can be selected from hyaluronic acid and its derivatives. In some embodiments, the hydrogel 400 can be selected from cellulose and its derivatives, such as hydroxypropyl cellulose, sodium carboxymethyl cellulose, etc. In some embodiments, the hydrogel 400 can be selected from natural macro-molecular proteins, such as silk fibroin. In some embodiments, the hydrogel 400 can be selected from synthetic polymers, including polyacrylamide (PAM) and its derivatives, such as NIPAM. In some embodiments, the hydrogel 400 can be selected from natural macromolecular proteins, such as silk fibroin. In some embodiments, the hydrogel 400 can be selected from amphoteric ion polymers, including gelatin-chitosan composites, chitosan-hyaluronic acid composites, chitosan-polyaspartic acid composites.

In some embodiments, the drug-loaded strip 100 contains very little hydrogel coating 400. In some embodiments, the drug-loaded strip 100 does not contain a hydrogel coating 400.

What is claimed:

1. A disposable male hydrogel patch, characterized in that it contains a backing, a drug-loaded strip, and may include a hydrogel coating; the backing adopts an ergonomic design with specific curvatures. The drug-loaded strip is arranged on the backing and divided into two zones: a central zone and side zones; the central zone encapsulates a first drug mixture that inhibits the dorsal nerve of the penis; the side zones contain a second drug mixture that promotes vasodilation related to erection; the hydrogel coating covers the drug-loaded strip and is used to control the sustained release of drug components.

2. The disposable male hydrogel patch according to claim 1, characterized in that the backing has specific curvatures designed to conform to the shape of the coronal sulcus for optimal fit and drug delivery.

3. The disposable male hydrogel patch according to claim 1, characterized in that the drug-loaded strip is divided into a central zone and side zones to enable targeted delivery of different drug mixtures to specific areas of the penis.

4. The disposable male hydrogel patch according to claim 1, characterized in that the right side of the backing has a tail end, and the tail end is provided with the adjustment hole for adaptively connecting to the left tail end of the backing.

5. The disposable male hydrogel patch according to claim 1, characterized in that the backing adopts a multi-layer composite material structure that integrates the functions of breathability, waterproofing, moisture absorption, and fixation.

6. The disposable male hydrogel patch according to claim 1, characterized in that the drug-loaded strip is prepared using materials with good elasticity, flexibility, hydrophilicity, adsorption capacity, and biocompatibility.

7. The disposable male hydrogel patch according to claim 1, characterized in that the first drug mixture contains herbal extracts such as Cnidium monnieri extract, clove extract, or synthetic anesthetics such as benzocaine or lidocaine.

8. The disposable male hydrogel patch according to claim 1, characterized in that the second drug mixture contains herbal extracts that promote erection or testosterone synthesis and metabolism, such as damiana extract, Rhodiola rosea extract, Erythroxylum extract, caffeine, and theophylline.

9. The disposable male hydrogel patch according to claim 1, characterized in that discrete protruding arrays are provided at the coronal sulcus recess of the drug-loaded strip, and the protruding arrays are hemispherical, semi-cylindrical, or semi-vertebral.

10. The disposable male hydrogel patch according to claim 1, characterized in that the hydrogel coating is prepared using natural polysaccharides, synthetic polymers, or amphoteric ion polymer materials.

11. The disposable male hydrogel patch according to claim 4, characterized in that the adjustment hole allow the left tail end to actively slide and adapt to the current size, accommodating changes in the circumference of the coronal sulcus brought about by the transition of the penis from a flaccid to an erect state.

12. The disposable male hydrogel patch according to claim 4, characterized in that the connection between the left tail end and the right tail end can be an embedded type, where the left tail end is directly inserted into a slit cut into the right tail end, or an externally embedded type, where the left tail end overlaps externally and is stitched to the right tail end.
